(19) United States
(12) Patent Application Publication
(10) Pub. No.: US 2002/0055495 A1
(43) Pub. Date: May 9, 2002

(54) Two-component anti-emetic composition

(76) Inventor: Anthony Jannetta, Haverford, PA (US)

Correspondence Address: PATENT ADMINSTRATOR, KATTEN MUCHIN ZAVIS, SUITE 1600, 525 WEST MONROE STREET, CHICAGO, IL, 60661, US

(21) Appl. No.: 09/946,173

(22) Filed: Sep. 5, 2001

Related U.S. Application Data
(60) Provisional application No. 60/229,547, filed on Sep. 5, 2000.

(51) Int. Cl. A61K 31/56; A61K 31/165

(52) U.S. Cl. 514/171; 514/619

(57) ABSTRACT

The invention relates to an anti-emetic composition containing dexamethasone (DEX) and metoclopramide (MTC). In a particular embodiment, a composition containing DEX:MTC in a relative weight ratio of about 1 to less than 1.25 is found to be effective in providing relief from the discomfort caused by symptoms of both vomiting and nausea in all patients receiving the composition. Alternatively, an effective suppository composition may contain DEX:MTC in a relative weight ratio of about 1:1-12.5. Other effective compositions and methods of their use are also disclosed.

### CROSS-REFERENCE TO RELATED APPLICATIONS

This application claims the benefit of an earlier-filed provisional application No. 60/229,547 filed Sep. 5, 2000, the entire disclosure of which is incorporated herein by reference.

### FIELD OF THE INVENTION

The present invention relates to both a therapeutic composition comprising a synergistic combination of antiemetic drugs and to a method for treating emesis, including nausea.

### BACKGROUND OF THE INVENTION

Nausea and vomiting can follow the administration of many drugs, particularly anticancer or chemotherapeutic agents. The symptoms also often accompany infectious and non-infectious gastrointestinal disorders.

The initial manifestations of the vomiting response often involves nausea, in which gastric tone is reduced, gastric peristalsis is reduced or absent and the tone of the duodenum and upper jejunum is increased, such that their contents reflux. Ultimately, the upper portion of the stomach relaxes while the pylorus constricts, and the coordinated contraction of the diaphragm and abdominal muscles leads to expulsion of gastric contents; see, for example, Goodman and Gilman's The Pharmacological Basis of Therapeutics, 8th Edition, Pergamon Press, New York, pp. 925-928 (1990).

The effects of various drugs in alleviating the symptoms of emesis have been studied. For example, in the Goodman and Gilman text, the authors mention metoclopramide (MTC), a benzamide, as a dopaminergic antagonist with important antiemetic uses. Benzodiazepines, another class of drugs, can enhance the effectiveness of antiemetic regimens and are thought to be beneficial in the prevention of anticipatory emesis. Also, dexamethasone (DEX) and other glucocorticoids or corticosteroids are said to have antiemetic effects and may improve the efficacy of antiemetic regimens in some cancer patients. The Goodman and Gilman authors name six phenothiazine compounds, one butyrophenone, two benzamides including metoclopramide and two cannabinoids as agents used in the treatment of nausea. Metoclopramide is described as being well tolerated in high intravenous dosages and being widely used to control emesis during cancer chemotherapy, especially when highly emetogenic agents, such as cisplatin or cyclophosphamide, are used. Metoclopramide has been combined with diphenhydramine (DPH). Regimens that are reportedly effective in countering vomiting induced by cisplatin or cyclophosphamide include those that utilize the intravenous administration of metoclopramide and dexamethasone in combination with lorazepam plus benztropine or droperidol plus diphenhydramine.

Various medical reports and issued patents exist wherein the above-named drugs were experimentally combined at various ratios to obtain an improved antiemetic effect. In the patented literature, such as U.S. Pat. No. 5,661,142, MTC is described as the agent of choice for suppressing emesis associated with cancer therapy, exhibiting an optimal antiemetic activity when combined with dexamethasone and diphenhydramine at 1:1:2.5 ratio. U.S. Pat. No.5,482,716, states that the antiemetic properties of carbazolone (1,2,3,9-tetrahydro-9-methyl-3-[(2-methyl-1H-imidazol-i-yl) methyl]-4H-carbazol-4-one) are enhanced by administering the compound in conjunction with DEX. In Example 6 of the U.S. Pat. No. 5,310,561, ondansetron is combined with MTC, haloperidol or droperidol, and DEX, among others.

Further examples of so-called “optimal” combination of MTC with other anti- emetics are found in abstracts of PubMed database. For example, Fujii et al. (Fujii Y et al., The effects of dexamethasone on antiemetics in female patients undergoing gynecologic surgery. Anesth Analg 1997 October;85(4):913-7) discloses the favorable effect when DEX is combined with MTC at 1:1.25 ratio. However these authors conclude that DEX-granisetron combination was more effective than DEX-MTC.

Du Bois et al. note that DEX:MTC ratio is optimal at 1:3 proportion. They conclude however that ondasetron plus DEX was significantly superior to MTC plus DEX (Du Bois A. et al., A randomized, double-blind, parallel-group study to compare the efficacy and safety of ondansetron (GR38032F) plus dexamethasone with metoclopramide plus dexamethasone in the prophylaxis of nausea and emesis induced by carboplatin chemotherapy. Oncology. 1997 January-February;54(1):7-14).

Soukop et al., while noting that DEX:MTC at 1:3.75 ratio was somewhat effective, have concluded that ondansetron was significantly superior than MTC (Soukop M et al., Ondansetron compared with metoclopramide in the control of emesis and quality of life during repeated chemotherapy for breast cancer. Oncology. 1992;49(4):295-304).

Several groups including Rath et al., Pollera et al., Strum et al., Shinkai et al., and Psilogenis combined DEX with MTC at about 1:5 ratio or higher. While they noted improved anti-emetic effect, they indicated that DEX in combination with other drugs was better or that higher doses of MTC were preferable (Rath U. et al. Role of ondansetron plus dexamethasone in fractionated chemotherapy. Oncology. 1993 May-June;50(3):168-72; Pollera C F et al., Alizapride alone or alizapride-dexamethasone compared with metoclopramide-dexamethasone in patients at high risk of acute emesis after cisplatin. A randomized cross-over study. Acta Oncol. 1991;30(6):725-9; Strum SB et al., High-dose intravenous metoclopramide versus combination high-dose metoclopramide and intravenous dexamethasone in preventing cisplatin-induced nausea and emesis: a single-blind crossover comparison of antiemetic efficacy. J Clin Oncol. 1985 February;3(2):245-51; Shinkai T et al., Antiemetic efficacy of high-dose intravenous metoclopramide and dexamethasone in patients receiving cisplatin-based chemotherapy: a randomized controlled trial. Jpn J Clin Oncol. 1986 September;16(3):279-87; WO 97/33573).

Other authors, e.g., Heron et al., and Chevallier tested DEX:MTC at higher 1:12.5 ratios, but have concluded that oral granisetron was superior to MTC (Heron J F et al., Oral granisetron alone and in combination with dexamethasone: a double-blind randomized comparison against high-dose metoclopramide plus dexamethasone in prevention of cisplatin-induced emesis. The Granisetron Study Group. Ann Oncol. 1994 September;5(7):579-4; Chevallier B. The control of acute cisplatin-induced emesis-a comparative study of granisetron and a combination regimen of high-dose metoclopramide and dexamethasone. Granisetron Study Group. Br J Cancer. 1993 July;68(1):176-80).

Thus, while numerous studies seem to exist, which have taught various ratios of DEX to MTC, none of these studies have been able to provide a dose range for the combination of DEX with MTC, which provides optimal anti-emetic effects. In most of these studies MTC was delivered intravenously. In most cases these studies effectively taught away from the DEX:MTC combination, as other drugs like ondansetron or granisetron in combination with DEX were apparently superior to MTC. Surprisingly, it has been discovered that DEX:MTC at ratio lower than 1:1.25 is the most effective anti-emetic combination and that enteral or topically formulated (suppository) delivery of MTC at even broader DEX:MTC ratios anywhere from about 1:1 to about 1:12.5) is more effective than the previously taught modes of intravenous administration.

### SUMMARY OF THE INVENTION

This invention relates to a method and a pharmaceutical composition for relieving nausea, emesis, or symptoms associated therewith. A pharmaceutical composition is provided for the relief of nausea, emesis, or symptoms associated therewith, which comprises a combination of dexamethasone (DEX) and metoclopramide (MTC). The components DEX and MTC are present in a relative weight ratio of about 1 to less than 1.25. Also, this composition can be made available in a form suitable for parenteral administration, e.g., intravenous, intraperitoneal, or intramuscular. Thus, both ingredients of the composition can be combined together in a variety of pharmaceutically compatible forms, i.e., a mixed solution for parenteral administration or a tablet with both ingredients mixed together or as an inhaled product.

A method is also provided wherein the anti-emetic composition of the invention is preferably administered orally or enterally. As oral administration in nauseous patients is often difficult, the composition is most preferably used in the form of a suppository, which is inserted into a patient's rectum, vagina, or otherwise administered enterally across a patient's mucosal membrane.

Although the composition of the invention can come in a number of forms and can be administered in a number of ways, the preferred composition is in the form of a two-component suppository. Both ingredients of the composition can be combined together in a single suppository. In this circumstance the anti-emetic suppository contains DEX to MTC weight ratio at about 1 to a range of MTC increments ranging between about 1 to about 12.5. Preferably, the DEX:MTC weight ration is 1:<1.25 more preferably, about 1:1.

A unit dosage form of the composition of invention will preferably have about 10 to about 20 mg of DEX and about 10 to about 20 mg of MTC. Even more preferably the unit dosage will have about 20 mg of DEX and about 20 mg of MTC. These dosages are particularly useful in a method of providing anti-emetic relief to a patient in need thereof. Preferably, the unit dosage form is administered one or more times daily. Even more preferably the unit dosage form is administered twice or three times daily. While mode of administration will depend on specific needs as determined by those of skill in the art the preferred mode of administration is parenteral. Another administration mode of choice is intranasal. Without limiting to these modes a sublingual administration is contemplated as well. Dosages can be equally well administered as a suppository.

### BRIEF DESCRIPTION OF THE DRAWING

FIG. 1 shows the effect of the addition of a dexamethasone on metoclopramide's locomotor depressant effects.

### DETAILED DESCRIPTION OF THE INVENTION

A suppository comprising 10 mg DEX and 10 mg MTC is compounded and administered as a suppository. Administration to affected patients provides relief from nausea. In this experiment, the dexamethasone is obtained from commercially available dexamethasone-containing tablets. The dexamethasone tablets are crushed using a mortar and pestle, and this powder is used to provide the desired content of dexamethasone. Commercial sources of powdered drug can also be used.

Indeed, a composition comprising 10 mg of finely divided dexamethasone dialkali phosphate salt, together with 10 mg MTC, is found to be very effective in providing relief against the discomfort caused by both nausea and vomiting. The composition is preferably administered in the form of a suppository. Preferably, the DEX is in the form of a micronized powder.

The clinical data is provided via the use and experience of a heterogeneous group of patients. Some have undergone, are undergoing, or will undergo chemotherapy. Still others are under the care of hospice groups. Most of these hospice group patients are terminally ill patients, including AIDS patients. Still other patients may have experienced a substantial or complete breakdown of their metabolic processes or capacity for metabolic function. Such breakdown may be the result of an advanced disease state, including but not limited to bacterial or viral infections one of whose pathological effects may be cell lysis, release of endogenous histamines and consequent inflammation.

In addition to the compositions described above, the invention contemplates other component mixtures, wherein DEX and MTC are admixed at ratios other than 1:1 or doses other than 10 mg:10 mg doses. These other ratios include a preferred composition for pediatric use having about 5 mg of DEX and about 5 mg of MTC. Still other embodiments may contain about 20 mg of DEX and about 20 mg of MTC per day as a single dose or divided doses. Alternative ratios for suppositories may comprise 8 mg of DEX and 10 mg of MTC, for example. These ratios can be anywhere in 1:1.25 to 12.5 ratios. For those patients who dislike the use of suppositories, other forms or routes of administration can be used. For example, these can be oral, parenteral, transdermal, or inhaled routes of administration as in incorporated by reference U.S. Pat. No. 5,192,528 which discloses art-accepted means of delivering corticosteroid is selected from the group consisting of aldosterone, beclomethasone, betamethasone, cloprednol, cortisone, cortivazol, deoxycortone, desonide, dexamethasone, difluorocortolone, fluclorolone, fluorocortisone, flumethasone, flunisolide, fluocinolone, fluocinonide, fluorocortolone, fluorometholone, flurandrenolone, halcinonide, hydrocortisone, meprednisone, methylprednisolone, paramethasone, prednisolone, prednisone and triamcinolone, and their respective pharmaceutically acceptable salts or esters.

The suppository can be formulated by conventional means and may contain a number of non-active components, such as cocoa-butter, polyethylene glycol, or glycerogelatin base. In addition to the three principal effective agents in the composition, stabilizers, bulking agents, emulsifiers, pH buffers and other therapeutic agents, known in the art, may be incorporated into the composition.

The composition of the invention can utilize a variety of dexamethasone derivatives, including esters thereof or salts thereof. In particular, the dexamethasone may be selected from the 21-(3,3-dimethylbutyrate), 21-phosphate, 21-phosphate dialkali metal salt, tetrahydrophthalate, 21-diethylaminoacetate, 21-isonicotinate, 21-(4-pyridinecarboxylate), 17,21-dipropionate, or 21-palmitate. Preferably, the dexamethasone is present as the phosphate salt, more preferably the dialkali or disodium phosphate salt and is most preferably the 21-phosphate disodium salt. Other corticosteroids are equally suitable including but not limited to hydrocortisone, triamcinolone and its derivatives, betamethasone and its derivatives, flunisolide, prednisone and its derivatives, fluocinolone and its derivatives, diflorasone and its derivatives, halcinonide, desoximetasone, diflucortolone and its derivatives, flucloronide, fluocinonide, fluocortolone, fluprednidene and its derivatives, flurandrenolide, clobetasol and its derivatives, clobetasone and its derivatives, alclometasone, flumethasone and its derivatives, and fluocortolone and its derivatives.

The preferred glucocorticoids include dexamethasone, fluorometholone, medrysone, betamethasone, triamcinolone, prednisone, prednisolone, hydrocortisone, and pharmaceutically acceptable salts thereof. Further examples of glucocorticoids include prednicarbate; deflazacort; halomethasone; tixocortol; prednylidene (21-diethylaminoacetate); prednival; paramethasone; methylprednisolone; meprednisone; maztpredone; isoflupredone; halopredone acetate; halcinonide; formocortal; flurandrenolide; fluprednisolone; flurprednidine acetate; fluperolone acetate; fluocortolone; fluocortin butyl; fluocinonide; fluocinolone acetonide; flunisolide; flumethasone; fludrocortisone; fluclorinide; enoxolone; difluprednate; diflucortolone; diflorasone diacetate; desoximetasone (desoxymethasone); desonide; descinolone; cortivazol; corticosterone; cortisone; cloprednol; clocortolone; clobetasone; clobetasol; chloroprednisone; cafestol; budesonide; beclomethasone; amcinonide; allopregnane acetonide; alclometasone; 21 -acetoxypregnenolone; tralonide; diflorasone acetate; deacylcortivazol; RU-26988; budesonide; and deacylcortivazol oxetanone. All of the above-cited glucocorticoids are known compounds. Further information about the compounds may be found, for example, in The Merck Index and the publications cited therein, the entire contents of which are hereby incorporated in the present specification by reference.

Metoclopramide can be present in the form of a free base or an acid addition salt. Metoclopramide (which is a N-substituted benzamide like sulpiride, tiapride, and alizapride), and other pharmacologically active N-substituted benzamides and phenothiazines are offered in commercial form as acid addition salts, presumably because this form is freely soluble in aqueous solution whereas the free base form is quite water insoluble. Hence, the acid addition salts of metoclopramide and other N-substituted drugs are pharmaceutically superior forms for bioavailability via a variety of routes of administration.

For example, the metoclopramide can be used as the dihydrochloride monohydrate (e.g., Emetid, Gastronerton, Primperan) or the monohydrochloride monohydrate (e.g., AHR-3070-C, Cerucal, Clopromate, Duraclamid, Emmeral, Gastrese, Gastrobid, Gastromax, Gastrosil, Gastro-Tablinen, Gastrotem, Gastro-Timelets, Maxeran, Maxolon, Meclopran, Metamide, Metoclol, Metocobil, Metramid, Moriperan, Mygdalon, Parmid, Paspertin, Peraprin, Plasil, Pramiel, Reglan). Equally suitable substitutes of MTC can be benzquinamide (at dose range 25-100 mg); meclizine (at dose range 25-100 mg); prochlorperazine (at dose range 5-25 mg); and trimethobenzamide (at dose range 100-2500 mg) among many others and as illustrative examples.

In one embodiment of the invention, a finely divided, micronized form of dexamethasone is employed in the form of the dialkali metal phosphate salt. The particle sizes of the micronized dexamethasone can range in size from about 5 μm up to about 20 μm. The composition is administered to a patient suffering from nausea, emesis or associated symptoms thereof. Once relief has been provided, the composition can be administered under a maintenance regimen to maintain a substantially symptom-free state. Generally, the dosage or frequency of administration of the composition of the invention to keep the patient essentially free of the complained afflictions will be less than the dosage or frequency used in the initial phase of treatment. The dosage or frequency can be cut back until the ailments begin to manifest themselves once again. The dosage or frequency is then adjusted to just suppress the symptoms.

The composition of the invention can thus be provided as part of a chemotherapeutic regimen with the benefit that the patient is better able to withstand the discomfort associated with same. The composition can be administered twice to three times daily. Thereafter, the composition can be taken once a day or less for maintaining a symptom-free state.

The composition of the invention is useful for providing relief to a patient experiencing an emetogenic condition. The present composition is particularly efficacious for treating patients undergoing, about to undergo, or recovering from chemotherapy for a deadly disease, such as cancer. However, other conditions, such as vertigo, motion sickness, AIDS, food poisoning and other acute or chronic diseases and infections that cause nausea, emesis, or associated symptoms thereof, may be effectively treated by the administration of the composition disclosed herein. In particular, the composition of the invention finds exceptional beneficial use in patients who have either exhausted all other medical alternatives or are considered terminally ill. In these patients (no matter what the cause of their illness) the composition provides exceptional relief of unwanted symptoms of nausea, vomiting and the like.

Moreover, it may be desirable to administer the present composition in the form of a controlled release formulation whereby constant uniform relief is provided over a predetermined period of time. This release may be accomplished by formulating the composition in a conventional vehicle for parenteral administration or encapsulating the composition in liposomes in a suppository and administering said suppository to the patient. By this method, relief from the debilitating effects of chemotherapy, cancer, immune deficiency and other potentially emetogenic conditions, including terminal or near terminal illnesses, may be provided and effectively maintained.

It is also pointed out that a range of 1-12.5 includes any amount within the recited range, as that amount relates to the amount of dexamethasone present in the composition, which dexamethasone amount is set to unity for the sake of comparison. In particular embodiments, the amounts of non-parenteral metoclopramide may be about 0.0, (i.e., substantially absent), 0.25, 0.5, 0.75, 1.0, 1.25, 1.5, 1.75, 2.0 or higher relative to dexamethasone.

Accordingly, the present invention provides a pharmaceutical product comprising metoclopramide and steroid such as dexamethasone as a combined preparation for simultaneous, separate or sequential use in the treatment and/or prevention of nausea and vomiting.

The present invention also provides a method of treatment and/or prophylaxis of nausea and vomiting, which comprises administering to a human or animal subject, metoclopramide and steroid such as dexamethasone or a pharmaceutically acceptable salt or ester thereof.

The invention further provides the use of metoclopramide for the manufacture of a medicament for administration in conjunction with steroid such as dexamethasone or a pharmaceutically acceptable salt or ester thereof, for the treatment and/or prevention of nausea and vomiting.

Co-administration of metoclopramide with steroid such as dexamethasone is particularly useful for the treatment and/or prevention of nausea and vomiting associated with chemotherapy using cytotoxic drugs, especially cancer chemotheraphy involving the use of, for example, platinum complexes such as cisplatin or carboplatin, or cyclophosphamide or doxorubicin. Such co-administration may also reduce delayed or prolonged nausea and vomiting associated with this type of chemotherapy. Particular note should also be made of the use in the treatment of nausea and vomiting associated with other cytotoxic agents, such as that associated with radiation treatment.

Metoclopramide and steroid such as dexamethasone or a pharmaceutically acceptable salt or ester thereof, may be administered as a single pharmaceutical composition comprising effective amounts of the two active ingredients. Alternatively the two active ingredients may be co-administered in the form of two separate pharmaceutical compositions for simultaneous or sequential use.

Suitable pharmaceutically acceptable salts of metoclopramide for use according to the invention include acid addition salts formed with organic or inorganic acids for example, hydrochlorides, hydrobromides, sulphates, phosphates, citrates, fumarates and maleates. The solvates may, for example, be hydrates. A preferred form of metoclopramide or sulpiride for use according to the invention is the hydrochloride.

Dexamethasone may be administered according to the invention as dexamethasone alcohol or in the form of a pharmaceutically acceptable salt or ester. Suitable salts and esters include the acetate, isonicotinoate, phenylpropionate, pivalate, t-butyl acetate, trioxaundecanoate, disodium metasulphobenzoate and disodium phosphate.

A proposed dosage of metoclopramide for use according to the invention for administration to man (of approximately 70 kg body weight), is 5 to 150 mg, more preferably 5 to 80 mg, and most preferably 5 to 20 mg per unit dose, expressed as the weight of free base. A preferred dose of steroid such as dexamethasone for use according to the invention is in the range of 0.5 to 20 mg per dosage unit, expressed as the weight of the alcohol.

The unit doses may be administered, for example, 1 to 4 times per day. The exact dose will depend on the route of administration and the condition being treated, and it will be appreciated that it may be necessary to make routine variations to the dosage depending on the age and weight of the patient as well as the severity of the condition to be treated. For convenience sake doses are preferably given one or more times per day. Ideally or alternatively doses are given twice or three times per day.

When the two active ingredients are administered as separate preparations, they are preferably given enterally, such as orally or as a suppository, or parenterally, e.g., intramuscularly or intravenously. Other modes like intranasal, sublingual, inhalation, transdermal patch, slow release forms, are well known in the art and are equally contemplated.

According to a further aspect the invention provides a pharmaceutical composition, for use in human or veterinary medicine, comprising the metoclopramide and steroid, such as dexamethasone or a pharmaceutically acceptable salt or ester thereof.

Compositions according to the invention may be formulated in conventional manner using one or more physiologically acceptable carriers or excipients. Thus the compositions may, for example, be formulated for oral, spray, buccal, parenteral or rectal administration. Compositions for administration by the oral route, in the form of for example tablets or capsules, are preferred.

Compositions for oral use such as tablets and capsules may be prepared by conventional means with pharmaceutically acceptable excipients such as binding agents (e.g. pregelatinised maize starch, polyvinylpyrrolidone or hydroxypropyl methylcellulose); fillers (e.g. lactose, microcrystalline cellulose or calcium hydrogen phosphate); lubricant (e.g. magnesium stearate, talc or silica); disintegrants (e.g. potato starch or sodium starch glycollate); or wetting agent (e.g. sodium lauryl sulphate). Tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g. sorbitol syrup, cellulose derivatives or hydrogenated edible fats); emulsifying agents (e.g. lecithin or acacia); non-aqueous vehicles (e.g. almond oil, oily esters, ethyl alcohol or fractionated vegetable oils); and preservatives (e.g. methyl or propyl-p-hydroxybenzoates or sorbic acid). The preparations may also contain buffer salts, flavoring, coloring and sweetening agents as appropriate.

Preparations for oral administration may be suitably formulated to give controlled release of one or both active ingredients.

For parenteral administration the compositions may be presented in a form suitable for bolus injection or continuous infusion. Formulations for injection may be presented in unit dosage form e.g. in syringes, ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredients may be in powder form for constitution with a suitable vehicle, e.g. sterile pyrogen-free water, before use.

For rectal administration the compositions may be formulated as suppositories or retention enemas, e.g. containing conventional suppository bases such as cocoa butter or other glycerides.

The pharmaceutical compositions of the invention may be prepared according to conventional techniques well known in the pharmaceutical industry. Thus, for example, the metoclopramide and the steroid such as dexamethasone or dexamethasone salt or ester may be admixed together, if desired, with suitable excipients. Tablets may be prepared, for example, by direct compression of such a mixture. Capsules may be prepared by filling the blend along with suitable excipients into gelatin capsules, using a suitable filling machine. Controlled release forms for oral or rectal administration may be formulated in a conventional manner associated with controlled release forms.

The compositions for use according to the invention may, if desired, be presented in a pack or dispenser device which may contain one or more unit dosage forms containing the active ingredients. The pack may for example comprise metal or plastic foil, such as a blister pack. The pack or dispenser device may be accompanied by instructions for administration. Where the metoclopramide and the steroid such as dexamethasone are intended for administration as two separate compositions these may be presented in the form of, for example, a twin pack.

### EXAMPLES

### Example 1

A randomized, double blind, parallel group study comparing the use of metoclopramide and/or dexamethasone for prophylactic control of cisplatin induced nausea and vomiting is carried out for a period of seven days. Randomization is stratified by cisplatin dose. Patients receive either placebo or metoclopramide and dexamethasone at 1:1 ratio for seven days. Drugs are given twice a day during first three days and then once a day for remaining days. The primary efficacy assessments in the study are the percentage of complete responders. Nausea and vomiting assessments after the first 24 hours provide clear evidence in efficacy of dexamethasone plus metoclopramide as compared to placebo.

### Example 2

A randomized, double blind, parallel group study is carried out, comparing once a day oral metoclopramide (12 mg p.o.) in combination with dexamethasone (10 mg p.o.) on the first day only and a conventional anti-emetic therapy (metoclopramide 10 mg/kg iv plus dexamethasone 12 mg iv) over a 7 day period in controlling cisplatin induced nausea and vomiting. The percentage of complete responders over the seven day period is significantly lower in the conventional mode than in the group receiving the inventive therapy.

### Example 3

A crossover clinical trial is carried out to compare the effectiveness and safety of oral metoclopramide (12 mg p.o.):dexamethasone (10 mg p.o.) with DEX:MTC combined in a suppository at a weight ratio of 1:12.5. Control of emesis and vomiting is achieved in both sets of patients, with the suppository patients faring slightly better than the patients receiving oral metoclopramide and dexamethasone. There are no clinical toxicities or side effects in either treatment group. This result indicates that quite a broad range, i.e., 1-12.5 of DEX:MTC is efficacious when the two drug combination is administered as a suppository.

### Example 4

This comparative study is undertaken to compare the efficacy and safety of metoclopramide plus DEX (15 mg doses of each drug per day) and same doses of metoclopramide plus methylprednisolone (MPL). One group of patients is given metoclopramide/DEX combination and another group of patients is given a combination of metoclopramide and MPL. Depending on the underlying clinical condition drugs are given one or more times per day, usually twice or three times per day. Half of the patients received the treatment in crossover fashion during the same chemotherapy regimens. The emetic and nausea episodes are counted during the 24 hours following each chemotherapy treatment. There is no significant differences in complete responses between the two groups, male and female, and each age group. These results indicate that any pharmaceutically acceptable corticosteroids are of about same efficacy when combined with MTC at select ratios. Other corticosteroids like prednisone, hydrocortisone, triamcinolone, betamethasone, flunisolide, fluocinolone, diflorasone, halcinonide, desoximetasone, diflucortolone, flucloronide, fluocinonide, fluocortolone, fluprednidene, flurandrenolide, clobetasol, clobetasone, alclometasone, flumethasone, fluocortolone, suitable derivatives, salts and esters thereof can also be used to advantage in the present invention.

### Example 5

Metoclopramide is an inhibitor of the dopamine D2 receptor which negatively effects motor function through D2 blockade causing extrapyramidal signs and symptoms (EPS). The effect of the addition of a steroid, such as dexamethasone, on metoclopramide's binding affinity at the dopamine D2 receptor is studied. The IC50 (the concentration that inhibits 50% of dopamine D2 binding) for metoclopramide is determined to be 8.6 nM. Dexamethasone is then added in relative concentrations based on weight to achieve the following results:

TABLE 1

| | | |
|---|---|---|
| Concentration | Weight Ratio | % Inhibition |

| DEX (nM) | MTC (nM) | DEX:MTC | D2 Binding |
|---|---|---|---|
| N/A | 8.6 | N/A | 50% |
| 0.7 | 8.6 | 1:12.5 | 9.33% |
| 3.4 | 8.6 | 1:2.5 | 19.00% |
| 6.9 | 8.6 | 1:1.25 | 17.6% |
| 8.6 | 8.6 | 1:1 | 10.6% |
| 17.2 | 8.6 | 1:0.5 | 6.3% |

The results show that in the presence of various concentrations of dexamethasone, inhibition of D2 binding is weaker than the control of metoclopramide alone, which achieved 50% inhibition at the concentration of 8.6 nM.

### Example 6

In this example, the effect of the addition of a steroid, such as dexamethasone, on metoclopramide's locomotor depressant effects is studied. Forty-eight male Spargue-Dawley rats (250-350 g) are used for this experiment. The rats are housed in pairs and allowed free access to food and water and are not handled except for cage changes and weighing prior to testing. All experimental procedures are performed during the rat's nocturnal cycle, in dimly lit behavior rooms, except that the rats are moved to adjacent behavior rooms 30 minutes prior to testing. Eight rats are tested at a time, four rats from each condition are tested together and groups are randomly chosen to control for order sequence effects.

The locomotor experiments are carried out in clear, acrylic 41×41×30 cm test chambers inside Ominitech Digiscan activity monitors (Accuscan, Inc.). Each monitor uses two arrays of infrared photocell beams (8×8 photocells, model RXYZCM-8) to detect several parameters of the rat's movement both horizontally and vertically. Data is collected in 5-min bins during testing.

Dexamethasone (Sigma) is dissolved in deionized water at concentrations of 0.16, 0.4, 0.8, and 4 mg/ml. Metaclopramide (Sigma) is also dissolved in deionized water at a concentration of 4.0 mg/ml (salt weight). Both drugs are prepared fresh the day of the experiment and all injections are given subcutaneously in the dorsal surface of the neck, in a volume of 1.0 ml/kg.

As shown in Table 2, each rat receives an injection of either saline or dexamethasone (0.16, 0.4, 0.8, and 4 mg/kg s.c.) in the home cage 15 minutes prior to receiving a second injection of either saline or metaclopramide (4.0 mg/kg s.c.). Each rat is then immediately placed into a locomotor testing box and the data is recorded for 30 minutes (5-min bins).

TABLE 2
Dosages, Ratios, and number of rats tested in each group.

Injection

| | | 1^{st} | 2^{nd} | |
|---|---|---|---|---|
| Ratios | | DEX | MTC | n |
| 1 | 25 | 0.16 | 4 | 8 |
| 1 | 10 | 0.4 | 4 | 8 |
| 1 | 5 | 0.4 | 4 | 8 |
| 1 | 1 | 4 | 4 | 8 |

| | | | |
|---|---|---|---|
| Control | saline | 4 | 8 |
| Control | saline | saline | 8 |

The data is analyzed with a one way ANOVA for the dependent variable, center distance traveled. Fisher's least significant difference tests (with an alpha set at 0.1 for statistical difference) is used to make specific comparisons between the groups.

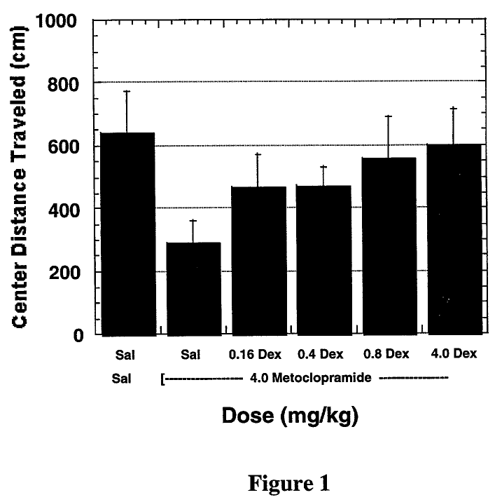

The results are shown in FIG. 1. The mean center distance traveled (cm) in rats receiving saline or dexamethasone (0.16, 0.4, 0.8, and 4 mg/kg s.c.) followed by a second injection of either saline or metaclopramide (4.0 mg/kg s.c.) is plotted. Error bars indicate standard error of the mean.

As shown in FIG. 1, metaclopramide (4 mg/kg) significantly reduces center difference traveled in rats pretreated with saline. However, pretreatment with various doses of dexamethasone reverses the locomotor depressant effects of metaclopramide. These findings demonstrate that pretreatment with various doses of dexamethasone dose-dependently reverses the locomotor depressant effects of metaclopramide.

The preferred embodiment of the invention and a few examples of its versatility are shown and described in the present disclosure. It is to be understood that the invention is capable of use in various other combinations and environments and is capable of changes or modifications within the scope of the inventive concept as expressed herein. Cited PubMed abstracts and patent literature are incorporated herein by way of reference.

What is claimed is:

1. An anti-emetic composition to be administered to a patient in need thereof, the composition comprising the combination of dexamethasone (DEX) and metoclopramide (MTC), wherein the weight ratio of DEX to MTC is about 1 to less than about 1.25.

2. The composition according to claim 1 wherein a total daily dose of DEX is about 10 mg per patient and a total daily dose of MTC is about 10 mg per patient.

3. The composition according to claim 1 wherein a total daily dose of DEX is about 20 mg per patient and a total daily dose of MTC is about 20 mg per patient.

4. The composition according to claim 1 wherein both DEX and MTC are oral compositions.

5. The composition according to claim 1 wherein both DEX and MTC are injectable compositions.

6. The composition according to claim 1 wherein DEX is an oral composition and MTC is an injectable composition.

7. The composition according to claim 1 wherein DEX is an injectable composition and MTC is an oral composition.

8. The composition according to claim 1 wherein said composition is administered twice a day.

9. The composition according to claim 1 wherein said composition is administered once a day.

10. The composition according to claim 1 wherein DEX and MTC are administered separately.

11. The composition according to claim 1 wherein DEX and MTC are administered simultaneously.

12. The composition according to claim 1 wherein DEX and MTC are mixed together and are administered as a single unit formulation.

13. The composition according to claim 1 wherein DEX and MTC are mixed together and are administered as a suppository.

14. A method of providing an anti-emetic relief to a patient in need thereof, said method comprising administering an effective amount of an oral pharmaceutical composition comprising the combination of dexamethasone (DEX) and metoclopramide (MTC), wherein the weight ratio of DEX to MTC is about 1 to about 1-12.5.

15. An anti-emetic suppository to be administered to a patient in need thereof, the composition comprising the combination of dexamethasone (DEX) and metoclopramide (MTC), wherein the weight ratio of DEX to MTC is about 1 to about 1-12.5.

16. The anti-emetic suppository of claim 15 comprising the combination of dexamethasone (DEX) and metoclopramide (MTC), wherein an amount of DEX in the suppository is in a range between 5 mg and 40 mg and an amount of MTC in the suppository is in a range between 5 mg and 150 mg.

17. An anti-emetic composition to be administered to a patient in need thereof, the composition comprising the combination of a corticosteroid and an N-substituted benzamide, wherein the weight ratio of the corticosteroid to the N-substituted benzamide, is about 1 to less than about 1.25.

18. The composition of claim 17 wherein the corticosteroid is selected from the group consisting of prednisone, hydrocortisone, tetrahydrocortisol, triamcinolone, betamethasone, flunisolide, fluocinolone, diflorasone, halcinonide, desoximetasone, diflucortolone, flucloronide, fluocinonide, fluocortolone, fluprednidene, flurandrenolide, clobetasol, clobetasone, alclometasone, flumethasone, fluocortolone, and salts or esters thereof.

19. The composition of claim 18 wherein the N-substituted benzamide, is selected from the group consisting of metoclopramide, sulpiride, tiapride, alizapride, emetid, gastronerton, primperan, AHR-3070-C, cerucal, clopromate, duraclamid, emmeral, gastrese, gastrobid, gastromax, gastrosil, gastro-tablinen, gastrotem, gastro-timelets, maxeran, maxolon, meclopran, metamide, metoclol, metocobil, metramid, moriperan, mygdalon, parmid, paspertin, peraprin, plasil, pramiel, reglan, benzquinamide, meclizine prochlorperazine, trimethobenzamide, and salts or esters thereof.

20. A unit dosage form of the composition of claim 1, having about 10 to about 20 mg of DEX and about 10 to about 20 mg of MTC.

21. The unit dosage of claim 20, having about 20 mg of DEX and about 20 mg of MTC.

22. A method of providing anti-emetic relief to a patient in need thereof comprising administering an effective amount of the composition claim 1.

23. A method of providing anti-emetic relief to a patient in need thereof comprising administering an effective amount of the unit dosage form of claim 20.

24. The method of claim 23 in which the unit dosage form is administered one or more times daily.

25. The method of claim 24 in which the unit dosage form is administered twice or three times daily.

26. The method of claim 22 in which the composition is administered parenterally.

27. The method of claim 22 in which the composition is administered intranasally.

28. The method of claim 22 in which the composition is administered sublingually.

29. The method of claim 22 in which the composition is administered as a suppository.
